(19) United States
(12) Patent Application Publication
(10) Pub. No.: US 2018/0280612 A1
(43) Pub. Date: Oct. 4, 2018

(54) EARLY WARNING BANDAGE FOR INTRAVENOUS BLOWOUT OR INTRAVENOUS INFILTRATION AND METHOD OF MONITORING AN INTRAVENOUS INFUSION SITE

(71) Applicant: Webb Medical LLC, Philadelphia, PA (US)

(72) Inventor: Steven B. Krupnick, Philadelphia, PA (US)

(21) Appl. No.: 15/806,849

(22) Filed: Nov. 8, 2017

Related U.S. Application Data
(60) Provisional application No. 62/481,295, filed on Apr. 4, 2017.

(51) Int. Cl. A61M 5/158

(52) U.S. Cl. CPC A61M 5/158; A61M 2205/18; A61M 2205/0216; A61M 2005/1586; A61M 2205/581; A61M 2205/3553; A61M 2205/8206

(57) ABSTRACT

Disclosed is an alarm bandage and method of use for monitoring an IV site of a patient. The bandage includes a first section, a second section, a bridge section, and associated electronic circuitry. The first section is adhesively secured to the skin of a patient on one side of the IV site. The second section is adhesively secured to the skin of the patient on the other side of the IV site. The bridge section is expandable and includes a breakable electronic link forming a portion of the electronic circuitry. The link is configured to break upon the expansion of the bridge section beyond a predetermined threshold, which will occur in the event that the vein at the IV site blows, whereupon the breakage of the link produces an alarm signal.

### CROSS-REFERENCE TO RELATED APPLICATIONS

This utility application claims the benefit under 35 U.S.C. § 119(e) of Provisional Application Ser. No. 62/481,295 filed on Apr. 4, 2017 entitled Early Warning Bandage for Intravenous Blowout or Intravenous Infiltration and Method of Monitoring an Intravenous Infusion Site. The entire disclosure of this provisional application is incorporated by reference herein.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISK

Not Applicable

### FIELD OF THE INVENTION

The disclosed invention relates to bandages and more particularly to bandages for monitoring an intravenous (IV) site.

### BACKGROUND OF THE INVENTION

A “blown vein” is a term used to describe what happens to a vein when it ruptures or gets punctured causing blood to leak outside of the vein itself. A vein may blow due to it being perforated by a needle that is too large or inserted incorrectly, or by a needle that is implanted too deeply into the vein causing both sides of the vein to be perforated and leading to possible blood leaking out of either end and potentially leading to infiltration or extravasation. Infiltration occurs when an intravenous fluid or medications leak into the surrounding tissue. There are many reasons why a vein blows. For example, some patients have inherently weak vein walls. There are also some specific age groups that are more prone to developing fragile veins. For example, geriatric patients commonly have weak vein walls due to age-related degeneration of connective tissues. Pediatric patients may also have weak vein walls which have not had sufficient time to develop.

In most cases a blown vein is not dangerous nevertheless when a vein blows it should be treated immediately since fluid infiltration can result in painful swelling of the surrounding tissue. If the medication being administered by the intravenous needle is caustic or otherwise dangerous, such as some chemotherapy drugs, dangerous extravasation can result in permanent damage to tissue surrounding the site of the blown vein.

The occurrence of a blow vein becomes more problematic if the patient is non-responsive or otherwise unable to communicate to his healthcare provider the fact that his/her vein has become blown. Thus, with those types of patients it is necessary to frequently check the status of their IV site. For example, it is common to check the IV site of an infant every thirty minutes or so to ensure that the IV hasn't blown and resulted in swelling. Needless to say, it is a waste of valuable medical resources to have to spend the time checking the status of an IV site. Accordingly, a need exists for a cost-effective means and method for automatically monitoring an IV site to detect the occurrence of a venous blow.

The subject invention addresses that need by providing a bandage which is simple in construction, low in cost, easy to use and which monitors the state of a venous needle access site to provide an alarm signal in the event of a venous blow.

### SUMMARY OF THE INVENTION

In accordance with one aspect of this invention there is provided an alarm bandage for use on a patient having an intravenous needle extending into a vein of the patient at an IV site. The bandage comprises a first section, a second section, a bridge section, and electronic circuitry. The first section is configured to be adhesively secured to the skin of a patient at a first securement location adjacent the IV site. The second section is configured to be adhesively secured to the skin of a patient at a second securement location adjacent the IV site. The bridge section interconnects and bridges the first and second sections and is expandable and configured to be expanded or stretched if the spacing between the first and second sections increases as a result of a venous blow causing the patient's body to swell at the location of the IV site or some other location. The electronic circuitry is mounted on the expandable section and configured to provide an electrical alarm signal in the event that the expandable section expands or stretches beyond a predetermined threshold.

In accordance with one preferred aspect of the bandage of this invention the electronic circuitry includes a breakable link configured to break or otherwise rupture in the event that the bridge section expands or stretches beyond the predetermined threshold, whereupon the alarm signal is provided.

In accordance with another preferred aspect the bandage of this invention one of the first section and the second section includes an electrical power supply for powering the electronic circuitry.

In accordance with another preferred aspect the bandage of this invention one of the first section and the second section includes an alarm producing member, e.g., a speaker, for producing the alarm signal.

In accordance with another preferred aspect the bandage of this invention the expandable section comprises an elastic cloth.

In accordance with another preferred aspect the bandage of this invention the electronic circuitry additionally comprises a separate unit including an electrical power supply and an alarm producing member, e.g., a speaker.

In accordance with another preferred aspect the bandage of this invention the separate unit is configured for wirelessly transmitting the alarm signal to a remote location.

Another aspect of this invention is a method of monitoring an IV site to provide an alarm signal in the event that the vein at the IV site becomes blown. The method comprises providing an alarm bandage. The alarm bandage includes a first section, a second section, and an expandable bridge section interconnecting the first and second sections. The bridge section includes electronic circuitry configured to provide an alarm signal in the event that the bridge section is expanded beyond a predetermined threshold. The first section of the bandage is adhesively secured onto the skin of a patient adjacent the IV site. The second section of the bandage is adhesively secured onto the skin of the patient adjacent the IV site, whereupon the bridging section is disposed over the IV site. Electrical power is applied to the electronic circuitry, whereupon in the event that the intravenous infusion results in the swelling of tissue at the IV site, the first and second sections move apart to stretch or expand the bridging section. If that stretching or expansion of the bridging section exceeds the predetermined threshold, the electronic circuitry provides an alarm signal.

In accordance with one preferred aspect of the method of this invention the alarm signal is an audible signal.

In accordance with another preferred aspect of the method of this invention the alarm signal is wirelessly transmitted to a remote location.

In accordance with another preferred aspect of the method of this invention the bridge section is pre-stretched to a point just below the predetermined threshold before the bandage is adhesively secured to the skin of the patient.

### BRIEF DESCRIPTION OF THE DRAWING FIGURES

FIG. 1 is top plan view of one exemplary embodiment of an alarm bandage constructed in accordance with one aspect of this invention, with the alarm bandage being shown mounted on the hand of a patient receiving an intravenous infusion to provide a warning in the event of an IV blow-out or infiltration;

FIG. 2 is a slightly enlarged top plan view of the bandage shown in FIG. 1;

FIG. 3 is a sectional view taken along line 3-3 of FIG. 2;

FIG. 4 is a top plan view similar to FIG. 1, but showing an alternative exemplary embodiment of an alarm bandage constructed in accordance with this invention and forming a portion of an IV blow-out or infiltration alarm system; and

FIG. 5 is a slightly enlarged top plan view of the bandage shown in FIG. 4.

### DETAILED DESCRIPTION OF A PREFERRED EMBODIMENT

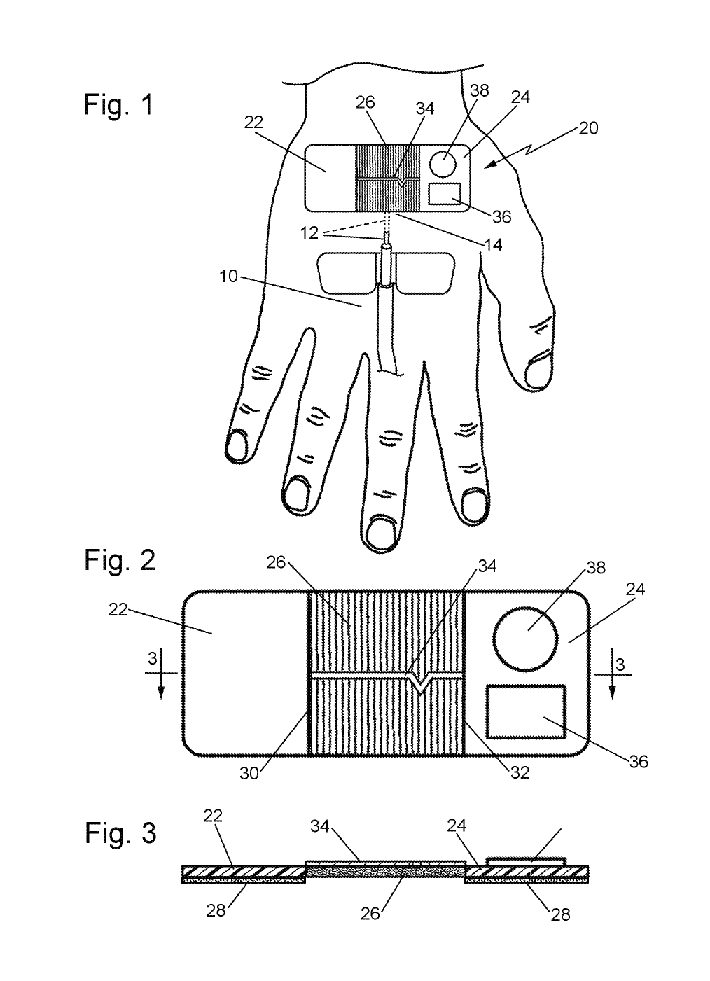

Referring now to the drawings wherein like characters refer to like parts, there is shown in FIG. 1 an exemplary embodiment of an alarm bandage 20 constructed in accordance with this invention shown in one typical application. The details of the bandage 20 will be described shortly. Suffice for now to state that the bandage 20 is configured to be adhesively secured to the skin of a patient over the IV site to automatically monitor the status of the IV site and provide an alarm signal in the event that the vein in which the needle extends blows. In the exemplary embodiment shown in FIG. 1, the bandage 20 is located on the back of the hand 10 of a patient into which an intravenous (IV) needle 12 extends, i.e., the IV site 14, and over the immediately adjacent area which will likely become swollen in the event that the vein into which the needle 12 extends blows.

The details of the bandage 20 are best seen in FIGS. 2 and 3. Thus, as can be seen, the bandage 20 basically comprises a first side section or patch 22, a second side section 24 and a bridge section 26. The first and second side sections are each formed of any suitable material, e.g., like that of a conventional adhesive bandage, or a knitted or woven fabric or a non-woven material. In the exemplary embodiment shown the side sections 22 and 24 are each formed of a patch of a woven fabric, plastic (PVC, polyethylene or polyurethane), or latex strip. The material making up the side section may or may not be waterproof and can be air-tight, if desired. The underside of each side section is in the form of a layer 28 of a conventional skin-engaging adhesive, such as but not limited to, acrylate, including methacrylates and epoxy diacrylates (which are also known as vinyl resins), or any other suitable adhesive materials.

The bridge section 26 is in the form of an expandable or stretchable web of any suitable elastic material, e.g., elastic cloth. The bridge section is fixedly secured to the first side section 22 at a joint 30 and is fixedly secured to the second side section 24 at a joint 32. A breakable link portion 34 of an electronic circuit (to be described shortly) is located on the bridge section 26. In the exemplary embodiment the portion 34 is in the form of a printed circuit, e.g., a link of electrically conductive ink. The link 34 is printed on and extends across the width of the bridging section 26 and is configured to break or rupture in the event that the bridging section expands to a point in excess of a predetermined threshold. The electronic circuit also includes a power source (e.g., a battery) 36, a loudspeaker 38, and associated circuitry (not shown) interconnecting the link, the battery and the loudspeaker to each other. The electronic circuit is configured to automatically provide an audible alarm signal from the loudspeaker 38 in the event that the link 34 ruptures or otherwise breaks.

Use of the bandage 20 is as follows. The bandage is applied to the skin of the patient at the location of the IV site 14 by pre-stretching the bridge section 26 to a point just below the point at which the conductive ink link 34 will rupture or otherwise break. With the bridge section pre-stretched, the bridge section is juxtaposed over the IV site and the adhesive underside 28 of the first side section 22 of the bandage is brought into engagement with the skin of the patient on one side of the IV site and the adhesive underside 28 of the second side section 24 of the bandage is brought into engagement with the skin of the patient on the other side of the IV site. Accordingly, the pre-stretched bridge portion 26 of the bandage will be located over the IV site 14, like shown in FIG. 1, and hence will be susceptible to being stretched or otherwise expanded in the event that the tissue at the IV site swells, as would occur if the vein into which the IV needle 12 extends should become blown. In that event, the additional stretching of the bridge section 26 will result in the rupture or breaking of the printed circuit link 34, whereupon the audible alarm signal will be produced by the loudspeaker

It should be pointed out at this juncture that the components making up the electronic circuitry, e.g., the printed link 34, the printed battery 36 and the printed loudspeaker 38 are merely exemplary of various components that can be used, in the bandage providing that some type of alarm signal is automatically produced by the circuitry in the event that the bridge section is stretched or otherwise expanded beyond a predetermined threshold. Moreover, the alarm need not be an audible alarm. Thus, the alarm can be a visual alarm. In fact, the alarm may be both audible and visual. In fact, the alarm (whatever type it is) need not be produced at the location of the bandage itself, but may be transmitted either wirelessly or otherwise to some remote location, e.g., a nurse's station, to alert personnel thereat that the IV site needs to be checked since it has likely blown.

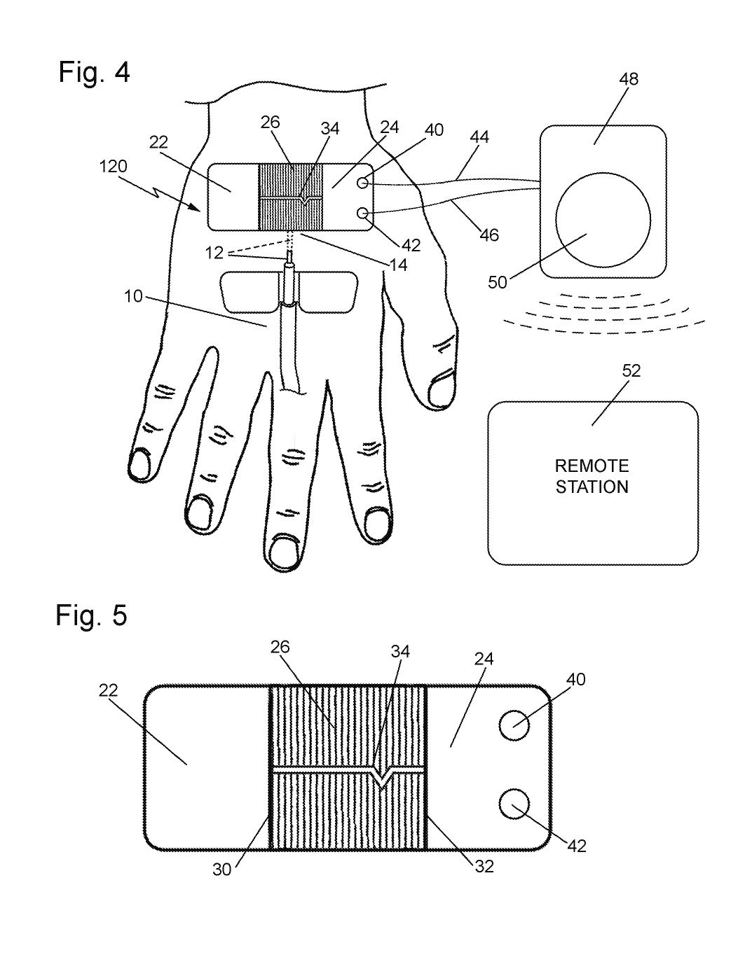

In FIG. 4 there is shown an alternative embodiment of an alarm bandage 120 constructed in accordance with this invention. The bandage 120 is identical in many respects to the bandage 20, except for the details of the electronic circuitry which provides the alarm signal. In the interest of brevity the components of the bandage 120 which are identical to the components of the bandage 20 will be given the same reference numbers and all the details of their construction and operation will not be reiterated. Thus, as can be seen in FIGS. 4 and 5 the bandage 120 basically comprises a first side section or patch 22, a second side section or patch 24 and a bridge section 26. The underside of each side section 22 and 24 is in the form of a layer 28 of a conventional skin-engaging adhesive. The bridge section is fixedly secured to the first side section 22 at a joint 30 and is fixedly secured to the second side section 24 at a joint 32. A breakable link portion 34 is printed on and extends across the width of the bridging section 26 and is configured to break or rupture in the event that the bridging section expands to a point in excess of a predetermined threshold. The side section 24 includes a pair of electrical connectors 40 and 42 which form portions of the electronic circuitry of the bandage. The connectors 40 and 42 are electrically connected to opposite sides of the breakable conductive link 34 by electrical conductors (not shown). The connectors 40 and 42 are configured to be connected to electrical conductors 44 and 46, respectively, of a separate unit 48. The unit 48 includes the remainder of the electronic circuitry of the bandage and basically comprises a housing in which a battery (not shown) for powering the electronic circuitry is located. The unit 48 also serves to house a loudspeaker 50. The loudspeaker is configured to provide an audible alarm in the event that the breakable link 34 ruptures or otherwise breaks, like described above. The unit 48 can be of an alternative construction. Thus, for example, the alarm provided by the unit 48 need not be an audible alarm, but can be a visual alarm or a combination of an audible and visible alarm. Moreover, the unit 48 may be constructed to transmit the alarm wirelessly or by some other means to a remote station 52.

It must be pointed out at this juncture that numerous other variations to the alarm bandages from those specifically disclosed above are contemplated. For example, the printed circuit (breakable) link 34 may be constructed so that it is micro-perforated, thereby making it easier to break when the bridge section is expanded. Moreover, the length of the side sections 22 and 24 of the bandage can be chosen for the particular anatomy to which they will be adhesively secured. For example, one or both of the side sections 22 and 24 can be considerably longer in length than the exemplary embodiments shown in FIGS. 1 and 3, so that one side section would be adhesively secured to the skin of the patient just laterally of the IV site, whereas the other side section would extend around the hand or arm of the patient (depending upon the location of the IV site) to the opposite side. That configuration should ensure that the bandage will not “float” on top of any swelling at the IV site. As should be appreciated by those skilled in the art if the bandage was to “float” on the swelling at the IV site the spacing between the side sections 22 and 24 will remain constant or not increase significantly, whereupon there will be little or no expansion of the bridge section and hence no alarm produced. Instead, with one side section of the bandage adhesively secured on one side of the IV site at a first location and with the other side section adhesively secured on the other side of the IV site at a second location which is more remote from the IV site than the first location, there will be a greater likelihood that in the event of an IV blow and swelling at the IV site, the spacing between the two side sections 22 and 24 will increase sufficiently, thereby resulting in the expansion of the bridge section and the concomitant breakage of the link 34, thereby resulting in the production of the alarm signal.

Without further elaboration the foregoing will so fully illustrate my invention that others may, by applying current or future knowledge, adopt the same for use under various conditions of service.

1. An alarm bandage for use on a patient having an intravenous needle extending into a vein of the patient at an IV site, said bandage comprising:
a first section configured to be adhesively secured to the skin of a patient at a first securement location adjacent the IV site;
a second section configured to be adhesively secured to the skin of a patient at a second securement location adjacent the IV site, said second location being spaced from said first location;
a stretchable bridge section interconnecting and bridging said first and second sections, said stretchable bridge section being configured to be stretched in automatic response to an increase in the spacing between said first and second sections as a result of a venous blow causing the patient's body to swell at the location of the IV site; and
electronic circuitry mounted on said stretchable bridge section and configured to provide an electrical alarm signal in the event that the spacing between the first and second sections causes said stretchable bridge section to stretch beyond a predetermined threshold.

2. The alarm bandage of claim 1 wherein said electronic circuitry includes a breakable link configured to break or otherwise rupture in the event that said bridge section expands or stretches beyond said predetermined threshold, whereupon said alarm signal is provided.

3. The alarm bandage of claim 1 wherein one of said first section and said second section includes an electrical power supply for powering said electronic circuitry.

4. The alarm bandage of claim 1 wherein one of said first section and said second section includes an alarm producing member for producing said alarm signal.

5. The alarm bandage of claim 3 wherein one of said first section and said second section includes an alarm producing member for producing said alarm signal.

6. The alarm bandage of claim 1 wherein said expandable section comprises an elastic cloth.

7. The alarm bandage of claim 1 wherein said electronic circuitry is comprises a printed circuit.

8. The alarm bandage of claim 3 wherein said electrical power supply is a printed battery.

9. The alarm bandage of claim 5 wherein said alarm producing member is a printed speaker.

10. The alarm bandage of claim 1 wherein said electronic circuitry additionally comprises a separate unit including an electrical power supply and an alarm producing member, said separate unit being configured to be releasably electrically connected to said alarm bandage.

11. The alarm bandage of claim 10 wherein said alarm bandage includes plural electrical connectors, and wherein said separate unit includes electrical wires, each of said wires having a respective electrical connector mounted thereon, said respective electrical connectors of said wires being configured to be connected to respective ones of said electrical connectors of said bandage.

12. The alarm bandage of claim 1 wherein said electrical alarm signal is provided wirelessly from said alarm bandage to a remote location.

13. The alarm bandage of claim 10 wherein said electrical alarm signal is provided wirelessly from said separate unit to a remote location.

14. A method of monitoring an IV site to provide an alarm signal in the event of a venous blow; said method comprising:
a) providing an alarm bandage comprising a first section, a second section and stretchable bridge section interconnecting said first and second sections, said stretchable bridge section being configured to be stretched in automatic response to an increase in spacing between said first and second sections, said stretchable bridge section including electronic circuitry configured to provide an alarm signal in the event that the bridge section is expanded beyond a predetermined threshold value;
b) adhesively securing said first section of said bandage onto the skin of a patient adjacent said IV site;
c) adhesively securing said second section of said bandage on the skin of the patient adjacent said IV site, whereupon said stretchable bridge section is disposed over the IV site; and
d) applying electrical power to said electronic circuitry, whereupon in the event that the intravenous infusion results in the swelling of tissue at the IV site, said first and second sections move apart to stretch said stretchable bridge section, and if the stretching of said stretchable bridge section exceeds said predetermined threshold value, said electronic circuitry provides an alarm signal.

15. The method of claim 14 wherein said alarm signal is an audible signal.

16. The method of claim 14 wherein said alarm signal is wirelessly transmitted to a remote location.

17. The method of claim 14 wherein said bridge section is pre-stretched to a value just below said predetermined threshold before said bandage is adhesively secured to the skin of the patient.
